(19) United States
(12) Patent Application Publication
(10) Pub. No.: US 2023/0225861 A1
(43) Pub. Date: Jul. 20, 2023

(54) MINIMAL FRAME PROSTHETIC CARDIAC VALVE DELIVERY DEVICES, SYSTEMS, AND METHODS

(71) Applicant: SHIFAMED HOLDINGS, LLC, Campbell, CA (US)

(72) Inventors: Claudio ARGENTO, Felton, CA (US); Ali SALAHIEH, Saratoga, CA (US); Connor MULCAHY, San Francisco, CA (US); Alice YANG, Campbell, CA (US); Thu Hoang PHAM, San Jose, CA (US); Hong DU, San Jose, CA (US); Troy THORNTON, San Francisco, CA (US)

(21) Appl. No.: 18/002,219

(22) PCT Filed: Jun. 16, 2021
(86) PCT No.: PCT/US21/37661

§ 371 Date: Dec. 16, 2022

Related U.S. Application Data
(60) Provisional application No. 63/039,909, filed on Jun. 16, 2020.

(51) Int. Cl. A61F 2/24

(52) U.S. Cl. CPC A61F 2/2418; A61F 2250/0039; A61F 2250/0069; A61F 2210/0076; A61F 2230/0004

(57) ABSTRACT

A device for treating a diseased native valve in a patient includes a frame structure and a valve segment coupled to the frame structure. The frame structure has an unexpanded configuration and an expanded configuration. The valve segment has a plurality of leaflets, a seal, and a seal support. An inflow edge of the plurality of leaflets is unsupported by the frame structure. The seal is attached to the inflow edge of the plurality of leaflets and positioned radially between the frame structure and the plurality of leaflets. The seal support is attached to or within the seal and provides axial rigidity to the seal.

### CROSS-REFERENCE TO RELATED APPLICATIONS

This application claims priority to U.S. Provisional Patent Application No. 63/039,909, filed on Jun. 16, 2020, titled “Minimal Frame Prosthetic Cardiac Valve Delivery Devices, Systems, and Methods,” the entirety of which is incorporated by reference herein.

This application may be related to International Application No. PCT/US2020/027744, filed Apr. 10, 2020, entitled “Minimal Frame Prosthetic Cardiac Valve Delivery Devices, Systems, and Methods,” the entirety of which is incorporated by reference herein.

This application may also be related to U.S. patent application Ser. No. 16/546,901, filed Aug. 21, 2019, entitled “Prosthetic Cardiac Valve Devices, Systems, and Methods; U.S. patent application Ser. No. 16/594,946, filed Oct. 7, 2019, entitled “Prosthetic Cardiac Valve Devices, Systems, and Methods”; International Patent Application No. PCT/US2019/057082, filed Oct. 18, 2019, entitled “Adjustable Medical Device”; U.S. patent application Ser. No. 16/723,537, filed Dec. 20, 2019, entitled “Prosthetic Cardiac Valve Devices, Systems, and Methods” and International Patent Application No. PCT/US2020/023671, filed Mar. 19, 2020, entitled “Prosthetic Cardiac Valve Devices, Systems, and Methods,” the entireties of which are incorporated by reference in their entireties.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transseptally, requiring transapical access instead. Furthermore, existing mitral valve replacement devices are not optimized with respect to strength-weight ratio and often take up too much space within the valve chambers, resulting in obstruction of outflow from the ventricle into the aorta and/or thrombosis.

Thus, a new valve device that overcomes some or all of these deficiencies is desired.

### SUMMARY

Described herein is a device for repair and/or replacement of heart valves, including the mitral valve, that is deliverable through minimally invasive techniques and that comprises a minimal amount of valve and/or stent material. Not necessarily all such aspects or advantages are achieved by any particular embodiment. Thus, various embodiments may be realized in a manner that achieves or optimizes one or more advantage or group of advantages taught herein without necessarily achieving other aspects or advantages as may also be taught or suggested herein.

The present disclosure generally relates to prosthetic heart valves for treatment or replacement of a diseased native valve in a patient and more particularly relates to prosthetic heart valves formed from a minimal amount of material and/or having a stiff region of minimal length.

The present disclosure generally relates to treating a diseased native valve in a subject, and more particularly relates to prosthetic heart valves.

In general, in one embodiment, a device for treating a diseased native valve in a patient includes a frame structure and a valve segment coupled to the frame structure. The frame structure has an unexpanded configuration and an expanded configuration. The valve segment has a plurality of leaflets, a seal, and a seal support. An inflow edge of the plurality of leaflets is unsupported by (e.g., unattached and/or unconnected to) the frame structure. The seal is attached to the inflow edge of the plurality of leaflets and positioned radially between the frame structure and the plurality of leaflets. The seal support is attached to or within the seal and provides axial rigidity to the seal.

This and other embodiments can include one or more of the following features. The inflow edge of the plurality of leaflets can be spaced radially inwards from an inflow edge of the frame structure when the frame structure is in the expanded configuration. The device can further include a nadir support skirt extending between the seal and an inflow edge of the frame structure. The inflow edge of the plurality of leaflets can extend axially beyond an inflow edge of the frame structure such that the inflow edge of the plurality of leaflets extends further in an inflow direction than the inflow edge of the frame structure. The seal support can be laminated within the seal. The seal can include polyurethane. The seal support can extend annularly within the seal. The seal support can include an undulating wireform. The seal support can extend proximate to the inflow edge of the valve segment. The seal support can extend closer to an inflow edge of the seal than an outflow edge of the seal. The seal support can be configured to pretension the seal. The seal support can be disconnected from the frame structure. The seal support can include a plurality of axial folds in the seal. Each axial fold can extend from an inflow edge of the seal to an outflow edge of the seal. The frame structure can have a longitudinal length of less than 35 mm in the expanded configuration. The frame structure can include a flared inflow section, a central annular section, and a flared outflow portion. The seal can be attached to the central annular portion. The flared inflow section and flared outflow section can be configured to engage an exterior anchor therebetween when the frame structure is in the expanded configuration. The leaflets of the plurality of leaflets can be attached to the frame structure only at commissures of the leaflets. At least portion of the inflow edge of the plurality of leaflets can extend axially beyond the frame structure while an entire outflow edge of the plurality of leaflets is positioned within the frame structure.

In general, in one embodiment, a device for treating a diseased native valve in a patient includes a frame structure, a valve segment coupled to the frame structure, and a nadir support skirt. The frame structure has an unexpanded configuration and an expanded configuration. The valve segment has a plurality of leaflets and a seal. An inflow edge of the plurality of leaflets is unsupported by (e.g., unattached and/or unconnected to) the frame structure. The seal is attached to the inflow edge of the plurality of leaflets and positioned radially between the frame structure and the plurality of leaflets. The nadir support skirt extends between the seal and an inflow edge of the frame structure.

This and other embodiments can include one or more of the following features. The nadir support skirt can extend from an inflow edge of the seal to the inflow edge of the frame structure. An inflow edge of the seal can be attached to the inflow edge of the plurality of leaflets. The inflow edge of the plurality of leaflets can be spaced radially inwards from an inflow edge of the frame structure when the frame structure is in the expanded configuration. The inflow edge of the plurality of leaflets can extend axially beyond an inflow edge of the frame structure such that the inflow edge of the plurality of leaflets extends further in an inflow direction than the inflow edge of the frame structure. The frame structure can have a longitudinal length of less than 35 mm in the expanded configuration. The frame structure can include a flared inflow section, a central annular section, and a flared outflow portion. The seal can be attached to the central annular portion. The flared inflow section and flared outflow section can be configured to engage an exterior anchor therebetween when the frame structure is in the expanded configuration. The leaflets of the plurality of leaflets can be attached to the frame structure only at commissures of the leaflets. At least portion of the inflow edge of the plurality of leaflets can extend beyond the frame structure while an entire outflow edge of the plurality of leaflets is positioned within the frame structure.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 shows a perspective view of an implantable valve prosthesis, in accordance with embodiments.

FIG. 2 shows a side view of the implantable valve prosthesis of FIG. 1 crimped, in accordance with embodiments.

FIG. 3 shows a side view of the implantable valve prosthesis device of FIG. 1 connected to an anchor, in accordance with embodiments.

FIG. 4 shows a perspective view of an implantable valve prosthesis, in accordance with embodiments.

FIG. 5 shows a perspective view of an implantable valve prosthesis, in accordance with embodiments.

FIG. 6 shows a perspective view of an implantable valve prosthesis, in accordance with embodiments.

FIG. 7A shows a side view of an implantable valve prosthesis with the valve segment extending proximal of the strut frame, in accordance with embodiments.

FIG. 7B shows a bottom view (i.e., from the outflow end) of the implantable valve prosthesis of FIG. 7A.

FIG. 8A shows a portion of a valve prosthesis, in accordance with embodiments.

FIG. 8B shows a bottom view of the valve prosthesis of FIG. 8A.

FIG. 8C shows a detailed side view of the valve prosthesis of FIG. 8A.

FIG. 9A shows a side view of a valve prosthesis, in accordance with embodiments.

FIG. 9B shows a bottom view of the valve prosthesis of FIG. 9A.

FIG. 10A shows a side view of an implantable prosthesis with minimal valve supports, in accordance with embodiments.

FIG. 10B shows a bottom view of the prosthesis of FIG. 10A.

FIG. 11 shows a perspective view of a valve prosthesis, in accordance with embodiments.

FIG. 12 shows a detailed side view of a valve prosthesis with minimal valve supports, in accordance with embodiments.

FIG. 13 shows a detailed side view of a valve prosthesis with minimal valve supports, in accordance with embodiments.

FIG. 14 shows a detailed side view of a valve prosthesis with minimal valve supports, in accordance with embodiments.

FIG. 15A shows a valve prosthesis having a seal with a seal support.

FIG. 15B shows the seal of FIG. 15A.

FIG. 16A shows a method of pretensioning a seal with a seal support.

FIG. 16B shows an exemplary effect of pretensioning a seal.

FIG. 17A shows a valve prosthesis with a nadir support skirt.

FIG. 17B shows a close-up of a portion of the valve prosthesis of FIG. 17A.

FIG. 18A is a schematic of a valve prosthesis with a seal support and a nadir support skirt as the valve is closed.

FIG. 18B is a schematic of the valve prosthesis of FIG. 18A as the valve is opened.

FIG. 18C is another schematic of the valve prosthesis of FIG. 18A as the valve is opened.

FIG. 19A shows an exemplary seal having axial folds therein.

FIG. 19B is a close-up of an axial fold of the seal of FIG. 19A.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present disclosure is described in relation to systems, devices, or methods for treatment or replacement of a diseased native valve of the heart, for example a mitral valve. However, one of skill in the art will appreciate that this is not intended to be limiting and the devices and methods disclosed herein may be used in other anatomical areas and in other surgical procedures.

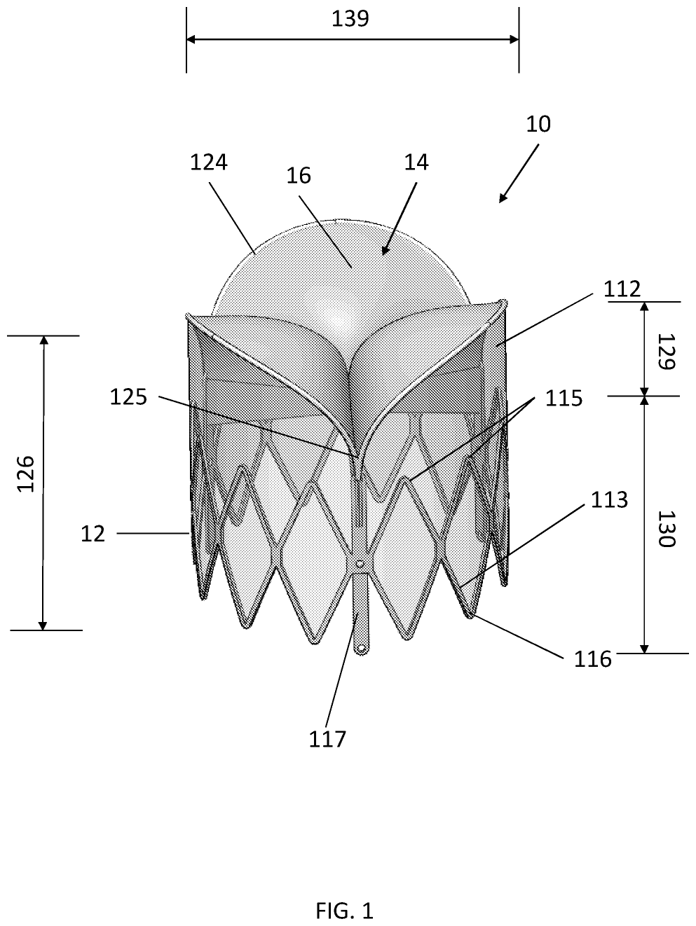

FIG. 1 shows a valve prosthesis 10 (e.g., an implantable valve prosthesis). The exemplary valve prosthesis 10 can include a frame structure 12 and a valve segment 14 positioned therein. Valve segment 14 can comprise a plurality of valve leaflets 16. In an expanded configuration, valve segment 14 can function as a fluidic valve in place of a native valve tissue (e.g., a heart valve, such as the mitral valve). The frame structure 12 can provide circumferential strength and/or longitudinal strength to valve prosthesis device 10.

One or more portions of valve prosthesis 10 can be shaped or configured to aid in securing valve prosthesis 10 at a location (e.g., in the orifice of a native heart valve). Described herein, for example, are various embodiments of anchors (e.g., spiral anchors 15) and flared portions (e.g., with flanges 159) that can aid in establishing or maintaining the valve prosthesis 10 at a location. In some embodiments, the valve prosthesis 10 can comprise one or more hook, barb, or scallop-shaped anchor to aid in deployment and/or positioning of valve prosthesis 10 at a location. In some cases, one or more hooks, barbs, or scallop-shaped anchor may be coupled to a portion of frame structure 12 (e.g., at a commissural post 117, a strut 113, a proximal arch 115, or a distal arch 116). For example, the frame structure 12 may comprise one or more hooks or barbs (e.g., connected to a strut 113), which can contact a tissue of a native heart valve or a tissue surrounding a native heart valve to prevent valve prosthesis 10 from moving or becoming dislodged from a location at which it has been placed or deployed.

FIG. 1 shows the valve prosthesis 10 in an expanded configuration. The valve prosthesis 10 can be deployed in an expanded configuration according to the methods described herein. For example, valve prosthesis 10 can be deployed into an expanded configuration in a method of replacing or repairing. In the expanded configuration, valve prosthesis 10 can be positioned and/or anchored at a target region of a subject (e.g., an organ or tissue of an animal such as a dog, cat, horse, or human). For example, valve prosthesis 10 can be positioned in the expanded configuration in the orifice of a heart valve, such as the mitral valve or tricuspid valve (e.g., to function as a temporary or permanent replacement for an existing mitral valve or tricuspid valve of the heart).

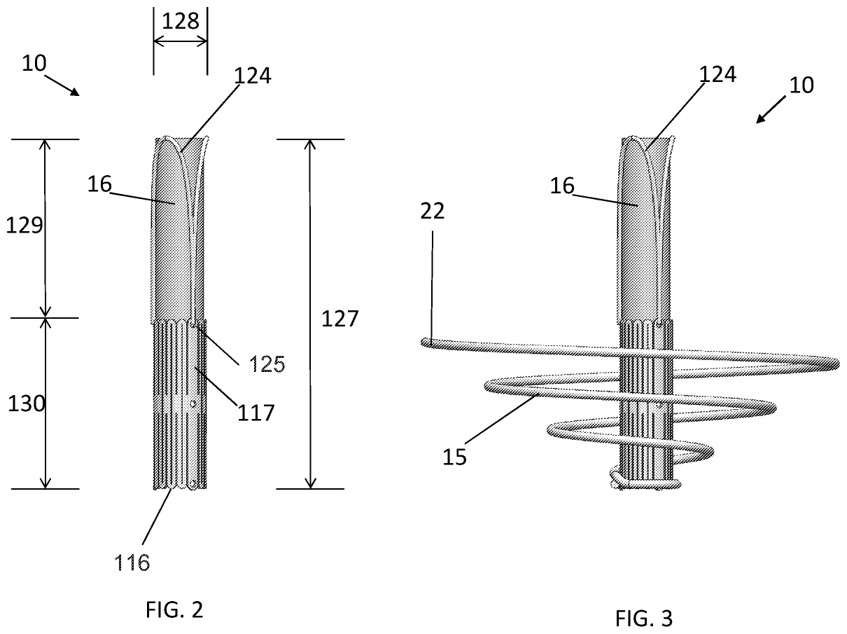

FIG. 2 shows the valve prosthesis 10 in an unexpanded (or collapsed or crimped) configuration. In some cases, the valve prosthesis 10 can be delivered to a target region (e.g., a region of a heart comprising a native valve) in the unexpanded configuration. In some cases, the valve prosthesis 10 in the unexpanded configuration can allow the valve prosthesis 10 to be delivered via minimally invasive means (e.g., via a delivery device, as described herein).

Referring to FIG. 2, the longitudinal length 127 of the collapsed valve prosthesis 10 can be minimized, which can be advantageous for delivery of the valve prosthesis 10. For example, minimizing the overall longitudinal length 127 of the collapsed valve prosthesis 10 can allow improved maneuverability within a delivery device while maintaining structural strength of the device. In some cases, minimizing the overall longitudinal length 127 of the collapsed valve prosthesis 10 can allow insertion of valve prosthesis 10 through an access path that would be challenging for a longer device to traverse (e.g., an access path comprising tortuous passages or passages with sharp turns). In some cases, the valve prosthesis 10 in the unexpanded configuration has an overall longitudinal length 127 of from 1 mm to 50 mm, from 1 mm to 45 mm, from 1 mm to 40 mm, from 1 mm to 35 mm, from 1 mm to 30 mm, from 1 mm to 25 mm, from 1 mm to 20 mm, from 1 mm to 10 mm, from 10 mm to 45 mm, from 20 mm to 45 mm, from 20 mm to 30 mm, from 25 mm to 35 mm, or from 27.5 mm to 32.5 mm. In some cases, the prosthetic delivery device 10 in the expanded In some cases, the prosthetic delivery device 10 in the expanded configuration can have an overall longitudinal length of from 1 mm to 45 mm, from 10 mm to 45 mm, from 15 mm to 45 mm, from 15 mm to 35 mm, from 16 mm to 34 mm, from 17 mm to 33 mm, from 18 mm to 32 mm, from 19 mm to 31 mm, from 20 mm to 30 mm, from 25 mm to 35 mm, or from 27.5 mm to 32.5 mm. In some embodiments, the valve prosthesis 10 can foreshorten as it expands such that the length 126 in the expanded configuration is less than the length 127 in the collapsed configuration.

Further, the diameter 128 of the collapsed valve prosthesis 10 can be minimized, which can likewise be advantageous for delivery of the valve prosthesis 10. For example, a collapsed valve prosthesis 10 with a smaller diameter 128 can fit inside of a delivery device with a smaller diameter, allowing for less invasive delivery and for improved maneuvering capability inside of a subject's body. Reducing the diameter 128 of the collapsed valve prosthesis 10 (e.g., for use in treatment or replacement of a mitral valve, a tricuspid valve, an aortic valve, or a pulmonic valve) can further allow for easier delivery of the valve prosthesis 10 to a target region of a subject, faster recovery of a subject receiving valve prosthesis 10, and/or improved clinical outcomes for a subject receiving valve prosthesis 10 (e.g., improved subject survival, improved ejection fraction, improved cardiac output, decreased valvular regurgitation, and/or decreased edema). In some cases, reducing the diameter 128 of the collapsed valve prosthesis 10 can make transseptal access and delivery possible in addition to transapical access. In some cases, the diameter 128 of the collapsed valve prosthesis 10 or portion thereof (e.g., frame structure 12) can be from 0.01 mm to 20 mm, 0.01 mm to 15 mm, 0.01 mm to 10 mm, from 0.01 mm to 9 mm, from 0.01 mm to 8 mm, from 0.01 mm to 7 mm, from 0.01 mm to 6 mm, from 0.01 mm to 5 mm, from 0.01 mm to 4 mm, from 0.01 mm to 3 mm, from 0.01 mm to 2 mm, from 0.01 mm to 1 mm, from 1 mm to 15 mm, from 2 mm to 14 mm, from 3 mm to 13 mm, from 4 mm to 12 mm, from 5 mm to 10 mm, from 6 mm to 10 mm, from 7 mm to 10 mm, from 8 mm to 10 mm, from 9 mm to 10 mm, from 10 mm to 15 mm, no more than 20 mm, no more than 15 mm, no more than 10 mm, no more than 9 mm, no more than 8 mm, no more than 7 mm, no more than 6 mm, or no more than 5 mm.

The diameter 139 of frame structure 12 in an expanded configuration (see FIG. 1) can be larger than the diameter 128 of frame structure 12 in an unexpanded configuration (see FIG. 2).

In some cases, frame structure 12 or a portion thereof (e.g., annular central portion 158 of frame structure 12) can have an expanded diameter 139 of from 10 mm to 50 mm, from 20 mm to 40 mm, from 25 mm to 35 mm, from 27 mm to 33 mm, no more than 50 mm, no more than 40 mm, no more than 35 mm, no more than 33 mm, no more than 30 mm, no more than 25 mm, no more than 20 mm, or no more than 15 mm when frame structure 12 is in an expanded configuration.

In some cases, the diameter 128 or 139 refers to a largest cross-sectional width of valve prosthesis 10 or a portion thereof, e.g., as measured in a plane perpendicular to a longitudinal axis of the valve prosthesis 10 at a longitudinal location. In some situations, the valve prosthesis 10 has a polygonal cross-section. In some cases, the diameter 128, 139 can refer to the largest distance from a first side of a polygonal cross-section of the valve prosthesis 10 to a second side of the polygonal cross-section of the valve prosthesis 10.

In some cases, the valve prosthesis 10 or a portion thereof can be sized or shaped to be positioned at a certain location or target region. For example, the frame structure 12 can be sized to be positioned in a valve, such as the mitral valve (e.g., by designing a dimension of frame structure to fit a valve, such as the mitral valve, when in an expanded configuration).

As shown in FIGS. 1-2, the valve prosthesis 10 can include a first portion 129 comprising only the valve segment 14 and/or minimal valve supports 124 and a second portion 130 comprising the frame structure 12 and the valve segment 14. In some embodiments, the valve segment 14 can be entirely unsupported or mostly unsupported in the first portion 129 while the valve segment 14 can be completely supported in the second portion 130 (e.g., by the frame structure 12). For example, minimal valve supports 124 can extend from the frame structure 12 to support the valve segment 14 in the first portion 129. The minimal valve supports 124 can, for example, support only the inflow edges of the valve segment 14 in the first portion 129 while leaving the rest of the valve segment 14 unsupported in the first portion 129. The first portion 129 of the valve prosthesis 10 can be coupled to or continuous with the second portion 130. For example, the frame structure 12 can be coupled to the minimal valve supports 124 at a joint 125 (e.g., with a fastener or crimp) or can be continuous with the minimal valve supports 124 (e.g., via fusion, welding, or formation by a continuous piece of material). Further, the valve segment 14 can be coupled to the minimal valve supports 124 in the first portion 129 and to the frame structure 12 in the section portion 130. When, for example, the valve prosthesis 10 is deployed in an orifice of the native mitral valve, the valve prosthesis 10 can be oriented such that the first portion 129 is positioned closer to the atrium than the second portion 130, and the second portion 130 can be positioned closer to the ventricle of the heart than the first portion 129.

FIG. 3 shows a representative example of the valve prosthesis 10 in an unexpanded configuration coupled to an anchor 15. In some embodiments, the anchor 15 may comprise a spiral shape that, for example, spirals around the valve prosthesis 10 in the unexpanded and/or expanded configuration. The anchor 15 can have a free end 22. In some cases, the free end 22 of anchor 15 can be useful during deployment of the anchor 15 in a native heart valve (e.g., by ensnaring chordae or other structures when the prosthesis 10, anchor 15, and/or delivery device are rotated around longitudinal axis of the valve prosthesis 10). The anchor 15 may be directly coupled to the frame structure 12, for example at a first end (e.g., a proximal end) or a second end (e.g., a distal end) thereof. Alternatively, the anchor 15 can be physically uncoupled from the frame structure 12 while providing an anchor for the frame 12 as the frame expands within the native valve orifice (thereby sandwiching tissue between the frame 12 and the anchor 15). In some embodiments, the frame structure 12 can be at least partially held in place within the native valve via interaction with the anchor 15. For example, the expanded diameter of the frame structure 12 can be greater than or equal to the inner diameter of the spiraled anchor 15 such that the frame structure 12 expands into and engages with the anchor 15 (with native valve leaflets, chordae, or other tissue therebetween).

A longitudinal axis of the anchor 15 may be co-axial or concentric with a longitudinal axis of the delivery device when the anchor 15 is in the deployed configuration. In some embodiments, the deployed anchor 15 may be detachably coupled to a delivery device prior to deployment of the valve prosthesis 10. For example, the anchor 15 can be deployed from a delivery device and held with a tether until the frame structure 12 is expanded within the native valve orifice and the anchor 15.

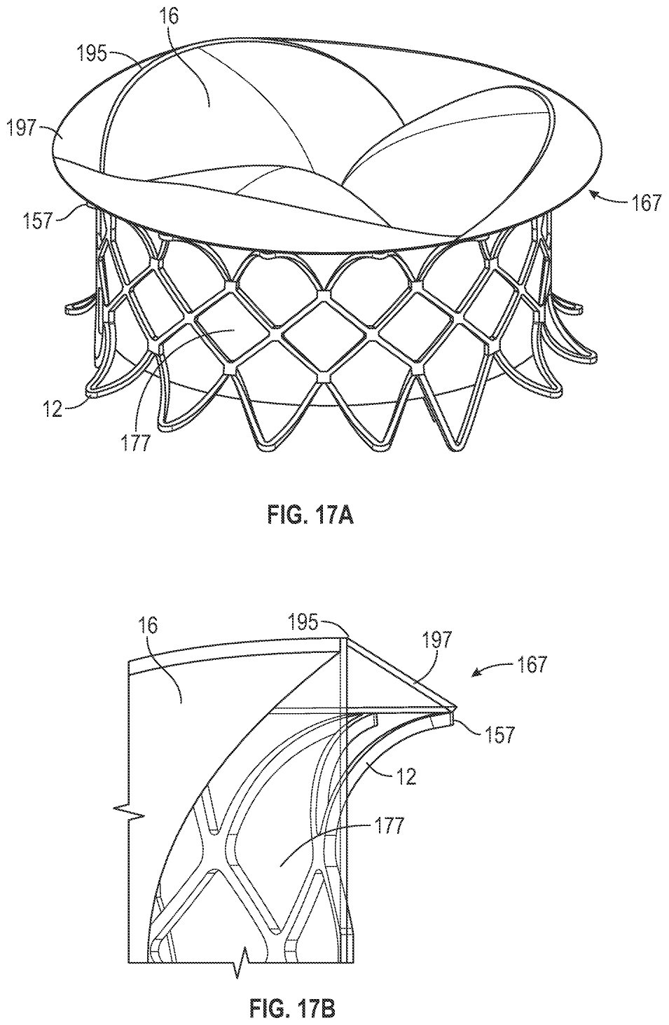

In some embodiments, the valve prostheses 10 described herein can include one or more flared portions to engage with the anchor 15 and/or help prevent the valve prostheses 10 from sliding through a valve orifice. For example, as shown in FIGS. 17A-17C, the frame structure 12 of can include an atrial flared portion 157 extending radially outwards from a central annular portion 158. The atrial flared portion 157 can, for example, extend into the atrium of the heart from the central annular portion 158 when valve prosthesis 10A is deployed in a native mitral valve. Alternatively, or in combination, the atrial flared portion 157 can contact a tissue of the atrium of the heart, e.g., a mitral valve annulus when valve prosthesis 10A is deployed in a native mitral valve.

The valve prostheses 10 described herein may comprise a first and second opposite ends, the first end (e.g., the proximal end) oriented nearest the atrium when the valve prosthesis 10 is deployed in the orifice of a native mitral valve and the second end (e.g., the distal end) oriented nearest the ventricle when the valve prosthesis 10 is deployed in the orifice of a native mitral valve. Alternatively, the frame structure 12 may be configured to sit entirely below the native valve when the frame structure 12 is anchored to the native valve. In some cases, a first portion of frame structure 12 can be disposed in a longitudinal location nearer to a first end of the valve prosthesis 10 than the second portion of frame structure 12 (e.g., when the frame structure is in an unexpanded configuration). A first portion and/or second portion of frame structure 12 can have a first longitudinal end and a second longitudinal end. In some cases, a first longitudinal end of frame structure 12 can be oriented nearer to a first end of valve prosthesis 10 than a second longitudinal end of frame structure 12. In some cases, a second longitudinal end of frame structure 12 is oriented nearer to a second end of valve prosthesis 10 than a first longitudinal end of frame structure.

Any of the frame structures 12 described herein can provide structural strength to valve prosthesis device 10. For example, the frame structure 12 can be used to anchor the valve prosthesis 10 in position at a target location of a subject (e.g., in the orifice of a heart valve, such as a mitral valve or tricuspid valve).

The valve prostheses 10 described herein may include one or more valve segments 14 disposed therein to replace the native valve leaflets. For example, the valve segment 14 can include a plurality of leaflets 16, e.g., that form a biocompatible one-way valve. Flow in one direction may cause the leaflets 16 to deflect open and flow in the opposite direction may cause the leaflets 16 to close.

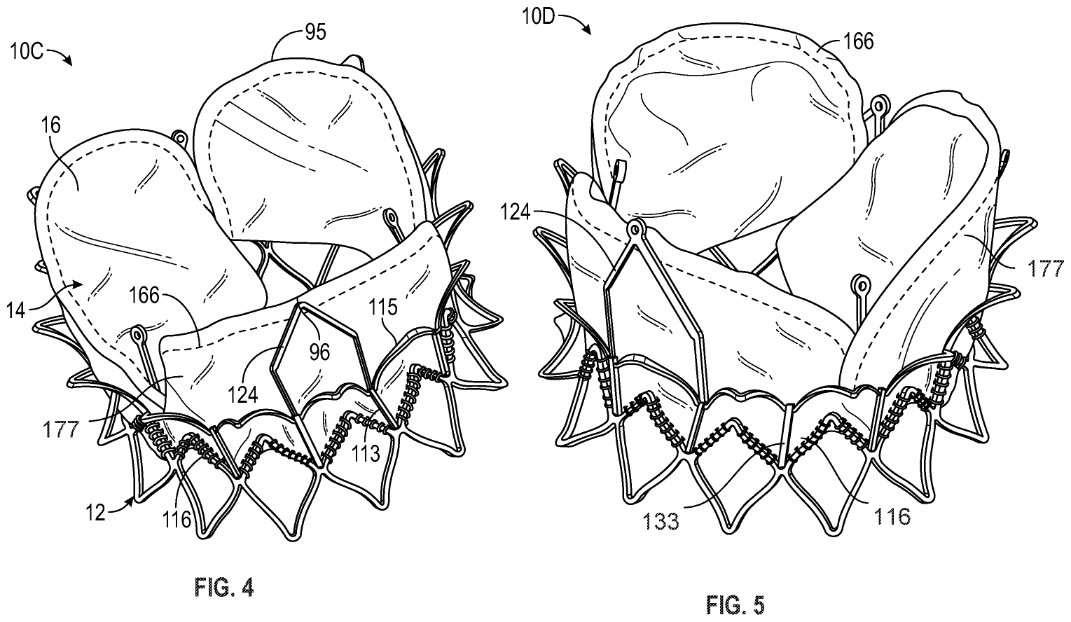

Any of the valve segments 14 described herein may be formed of multi-layered materials for preferential function. Referring to FIG. 4, for example, the valve prosthesis 10C may include a valve segment 14 having a seal 177 (also called an outer leaflet, outer layer, or skirt) positioned radially between leaflets 16 (also called inner leaflets or the inner layer) and the frame structure 12. The seal 177 can be a single piece wrapped around the leaflets 16 or can be individual pieces shaped to match the leaflets 16. In some cases, the seal 177 and/or leaflets 16 can be formed from or coated with a material to confer an advantage upon the valve segment 14. For example, a layer or surface of a valve segment 14 can be formed from or coated with a biocompatible material. In some cases, a layer or surface of a valve segment 14 can be formed from or coated with an anti-thrombotic material. In some cases, a valve segment 14 (or portion thereof, such as a leaflet 16 of the valve segment) comprises a synthetic material. In some cases, a valve segment 14 (or portion thereof, such as a leaflet) comprises a biological tissue. In many cases, a valve segment 14 (or portion thereof, such as a leaflet) comprises pericardial tissue. In some embodiments, a valve segment 14 (or portion thereof, such as a leaflet 16 of the valve segment 14) comprises a decellularized biological tissue. For example, a valve segment 14 (or portion thereof, such as a leaflet 16 of the valve segment) can include decellularized pericardium.

The valve segment 14 may be attached to a frame structure 12, which can in turn be attached to the anchor 15. The frame structure 12 may be connected to the anchor 15 before or after the frame structure 12 has been deployed adjacent a native valve. The frame structure 12 may be attached to the valve segment 12, for example, via attachment of the frame structure 12 to the seal 177, which can in turn be attached to the leaflets 16.

In some embodiments, two or more portions of a valve segment 15 (e.g., two or more leaflets 16, and/or seal 177) can comprise a single piece of material (e.g., a single piece of biological or synthetic tissue formed into the shape of a functional valve). In some cases, two or more portions of a valve segment (e.g., two or more of a first and second leaflet 16, and/or the seal 177) can be joined together. In some embodiments, two or more portions of a valve segment (e.g., two or more of a first and second leaflet 16, and/or the seal 177) can be joined together by suturing the two or more portions together (e.g., at sutured coupling 166 shown in FIG. 4). In some cases, 1, 2, 3, 4, 5, or more than 5 leaflets 16 can be coupled to a single seal 177.

In many cases, leaflet coupling 166 is disposed at an inflow end of valve prosthesis 10 (i.e., closest to the source of flow through the device, e.g., caused by a contracting heart chamber) when deployed. In some cases, coupling two or more portions of a valve segment 14 at the inflow end of valve prosthesis 10 (or portion thereof) allows the valve segment 14 to fold or collapse (e.g., radially away from a longitudinal axis of valve prosthesis device 10) during contraction of a heart chamber upstream of the deployed device (i.e., during diastole). Further, in some cases, coupling two or more portions of a valve segment 14 at the inflow end of valve prosthesis 10 causes the valve segment 14 to expand (e.g., radially toward a longitudinal axis of valve prosthesis device 10) during refilling of a heart chamber upstream of the deployed device (i.e., during systole). This expansion of the valve segment 14 can, for example, result in billowing or parachuting of the valve segment 14 (e.g., between the seal 177 and the leaflets 16) to block the flow of blood therethrough.

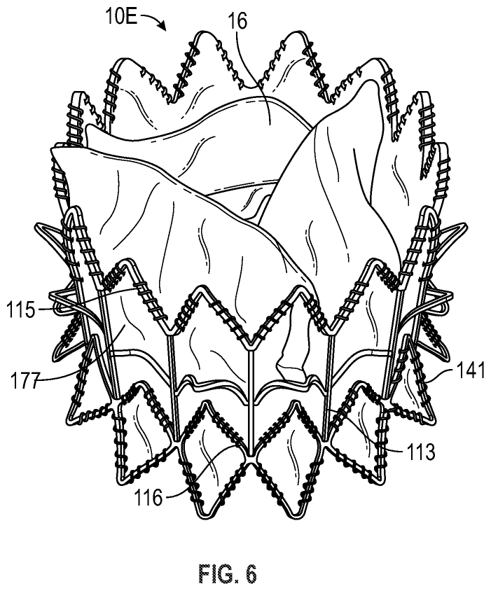

As shown in to FIG. 4, the valve segment 14 can be attached to one or more struts 113 of the frame structure 12. In some embodiments, a portion of a valve segment 14 (e.g., leaflets 16 or seal 177) can be sutured to the central annular portion 158 of frame structure 12 and not to the inflow portion of frame structure 12 or the outflow portion of frame structure 12 (e.g., can be unattached to the distal arches 116 and the proximal arches 115 as shown in FIG. 4). In some embodiments, a portion of a valve segment 14 (e.g., leaflets 16 or seal 177) can be sutured to one or more outflow portion of frame structure 12 and not to the inflow portion of frame structure 12 (e.g., can be sutured to one or more distal arches 116 but not one or more proximal arches 115 as shown in FIG. 9A). In some embodiments, a portion of a valve segment 14 (e.g., leaflets 16 or seal 177) can be sutured to one or more outflow portion of frame structure 12 and to the inflow portion of frame structure 12 (e.g., can be sutured to one or more distal arches 116 and also to one or more proximal arches 115 as shown in valve prosthesis 10E of FIG. 6). In some embodiments, an inflow end of the valve segment 14 can be substantially unsupported by the frame 12 while the outflow end of the valve segment 14 can be fully supported by and within the valve segment 14 (as shown in FIG. 4). The valve segment 14 (or portion thereof, such as the seal 177) can be coupled to the frame 12 continuously around the inner circumference of the frame 12 (e.g., at a distal or outflow end of valve prosthesis device 10).

In some cases, the amount of attachment of a valve segment 14 (e.g., a valve leaflet 16) to the frame structure 12 can be minimized, which can advantageously enhance ease of delivery and reduce the required length of the frame, thereby reducing the chance of thrombosis and reducing the chance of blocking the outflow from the ventricle to the aorta. Minimizing the frame structure 12 can also improve the speed and cost of fabrication of the valve prosthesis device 10.

In some embodiments, a leaflet 16 that is attached to a first portion of frame structure 12 (e.g., one or more struts 113) at a distal end of frame structure 12 can be unattached at a proximal end of the frame structure 12 (e.g., a strut or portion thereof at a proximal end of frame structure 12). In some cases, valve prosthesis devices 10 in which a valve segment 14 is attached at a proximal end of frame structure 12 and is unattached at a proximal end of frame structure 12 (and/or at a proximal end of valve segment 14) may require less metal and/or fewer struts than a valve prosthesis 10 in which a valve segment 14 is attached at both a proximal end and a distal end of the frame structure 12 of the valve prosthesis device 10. In some cases, minimizing the amount of metal used in the structure of valve prosthesis 10 (e.g., by reducing the number and/or length of struts in valve prosthesis device 10) can reduce the risk of thrombus formation and can improve the ease with which the device is deployed at a target location.

Further, the valve segment 14 can be configured to be substantially unsupported at the inflow edge 95 of the valve segment 14. For example, as shown in FIG. 4, the entire inflow edge 95 of valve segment can be unsupported with the exception of minimal valve supports 124 positioned at the nadir 96 of each leaflet 16. The valve supports 124 can have a pointed proximal tip and can extend, for example, from two neighboring struts 113 of the frame structure 12. The minimal valve supports 124 can help prevent the valve segment 14 (e.g., the seal) from collapsing radially inwards in the outflow direction (i.e., towards the ventricle) when implanted in the heart. FIG. 5 shows a valve prosthesis 10D that is similar to valve prosthesis 10C of FIG. 4 except that the valve support 124 of FIG. 5 includes an aperture 97 for suturing the leaflet 16 to the valve support 124.

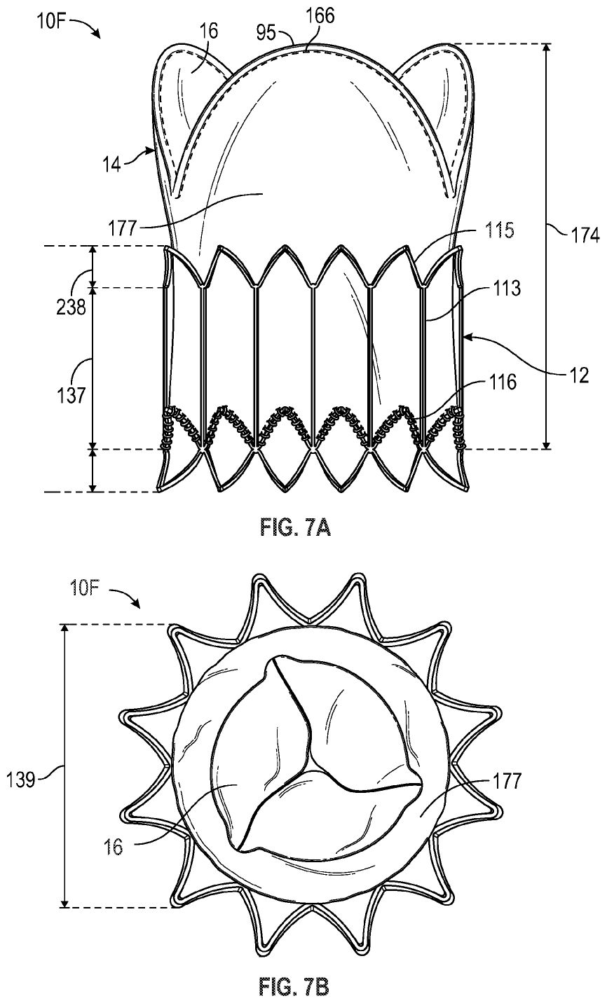

FIGS. 7A-7B show a valve prosthesis 10F wherein the inflow edge 95 of valve segment 14 is completely unsupported (i.e., does not include any valve supports thereto).

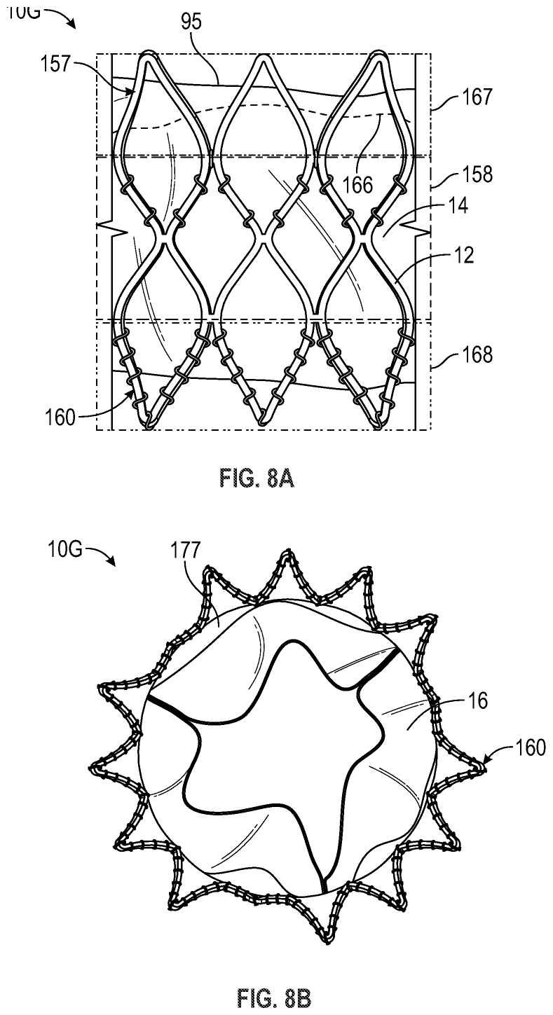

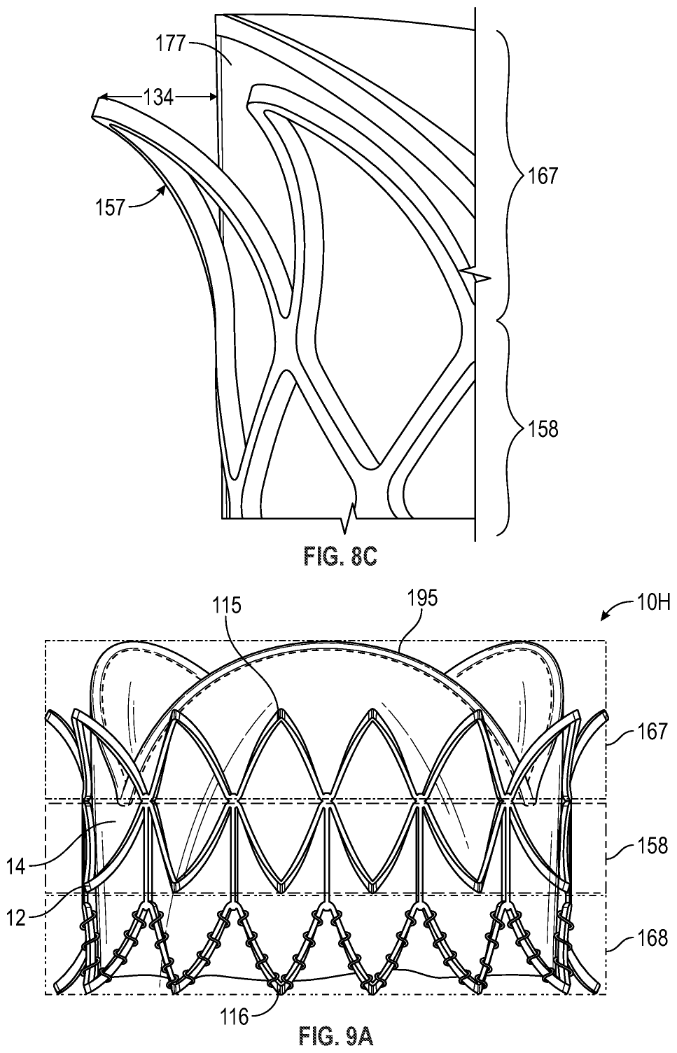

FIGS. 8A-8C show another valve prosthesis 10G wherein the inflow edge 95 of valve segment 14 is completely unsupported (i.e., does not include any valve supports thereto). Indeed, as shown in FIGS. 8A-8C, the prosthesis 10G can include an inflow portion 167, a central annular portion 158, and an outflow portion 168. The valve segment 14 can be fully circumferentially supported by the frame structure 12 within the central annular section 158. However, the valve segment 14 can be unsupported by and/or unconnected from the frame structure 12 in the inflow section 167. Further, the frame structure 12 can flare radially outwards within the inflow section 167. The flared portion 157 of the frame structure 12 can include a plurality of discrete flanges (i.e., formed from flared proximal arches 115) and can, for example, serve to help engage with an external anchor. Moreover, due to the flared portion 157, the valve segment 14 can be radially spaced away from the frame structure 12 within the inflow section 167 by a distance 134 (see FIG. 8C). In some embodiments, the distance 134 can be 1-10 mm, such as 2-8 mm, such as 3-5 mm. Finally, the frame structure 12 can also flare radially outwards within the outflow section 168. The flared portion 160 of the frame structure 12 can also serve to help engage with an external anchor 15. For example, the external anchor 15 can sit between the flared portions 157, 160 upon implantation.

FIG. 11 shows another valve prosthesis 10J that is similar to valve prosthesis 10G of FIGS. 8A-8B except that the ratio of width of the cells (i.e., in the circumferential direction) to height of the cells can be greater in valve prosthesis 10J than valve prosthesis 10G. The dimensions of the cells can be modified to provide the desired stiffness and stability.

FIGS. 9A-9B show another valve prosthesis 10H that is similar to valve prosthesis 10G of FIGS. 8A-8C except that substantially all of the inflow edge 95 extends proximally beyond the proximal arches 15 of the frame structure 12. When the leaflets are closed (as shown in FIG. 9B), the fluid pressure can act to fill the space created by the leaflets 16 and the seal 177, thereby preventing inward motion or collapse of the valve segment 14.

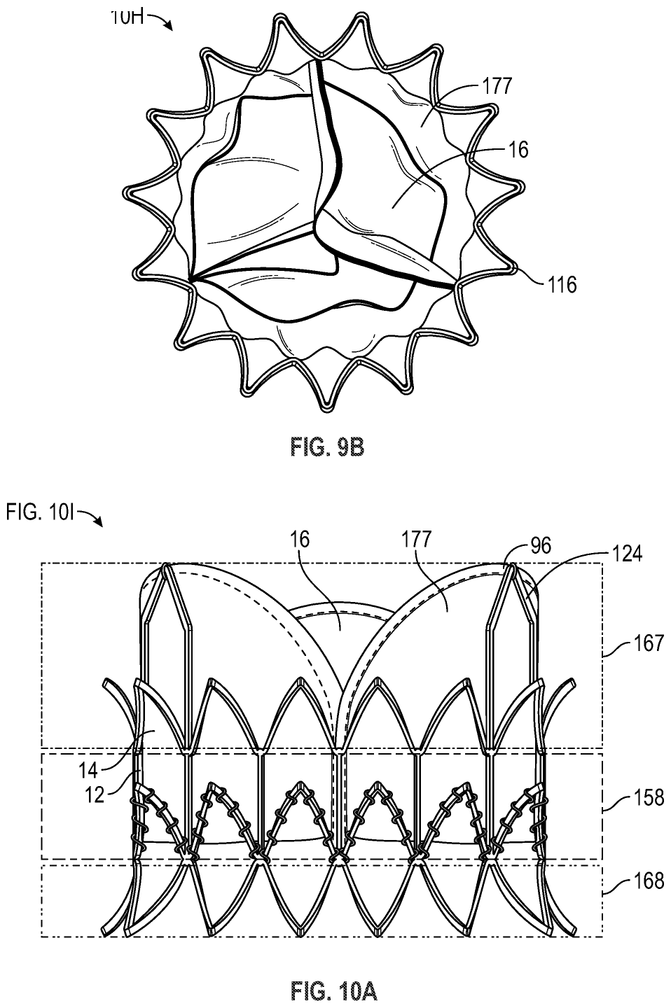

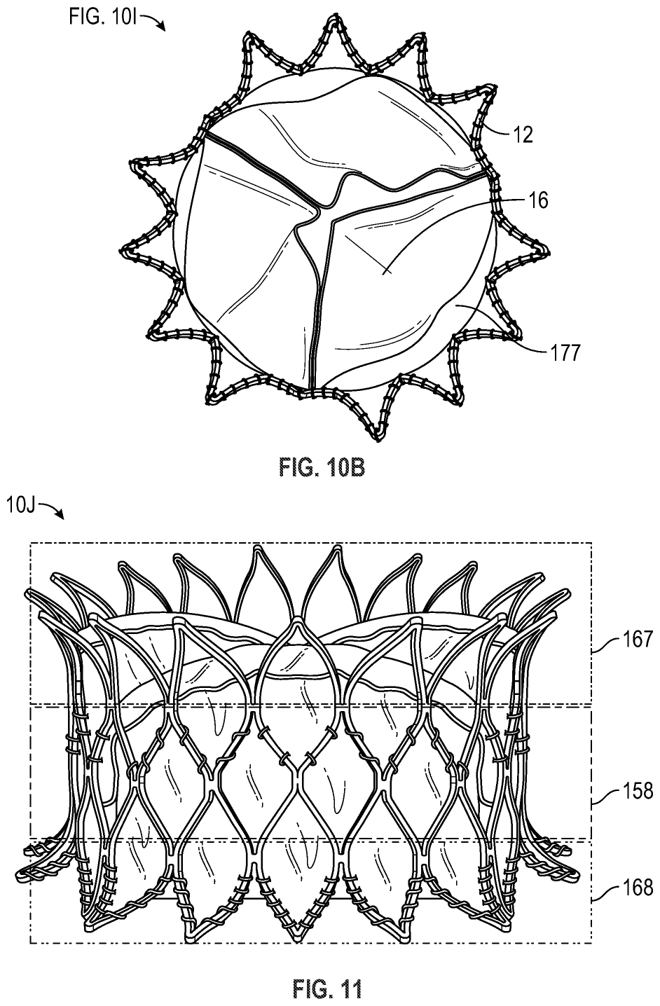

FIGS. 10A-10B show a valve prosthesis 10I that is similar to valve prosthesis 10H of FIGS. 9A-9B except that it includes a minimal valve support 124 at the nadir 96 of each leaflet 16. The minimal valve supports 124 are similar to the valve supports 124 of FIG. 4. Additionally, the valve segment 14 of valve prosthesis 10I ends before the start of the outflow section 167 (i.e., ends within the central annular section 158). Not having the valve segment 14 attached at the outflow section 167 may advantageously reduce tension on the frame structure 14 where the frame structure 14 engages the external anchor 15 (i.e., within the outflow section 167).

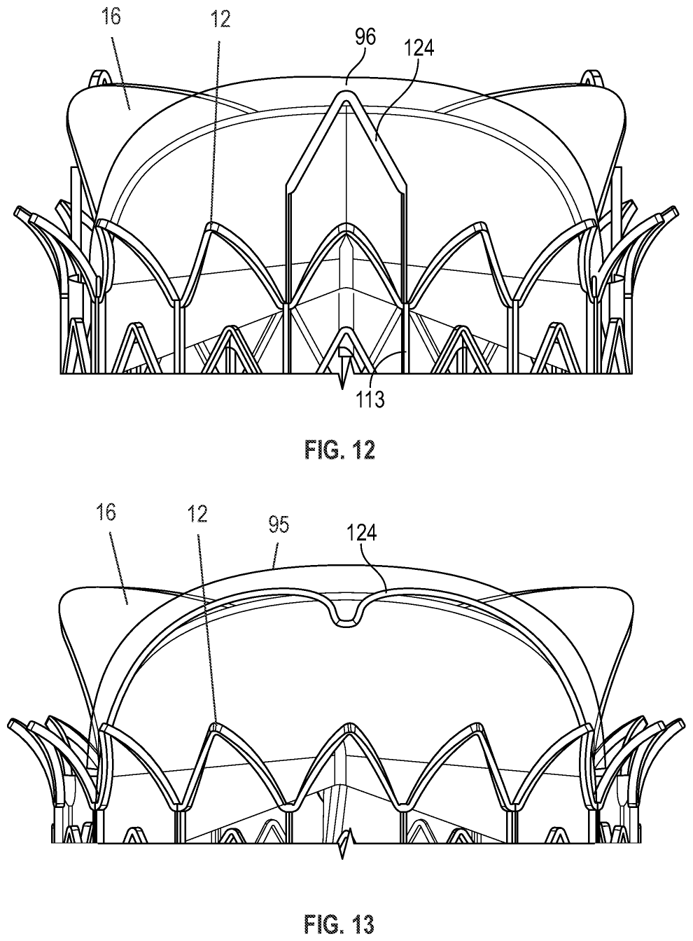

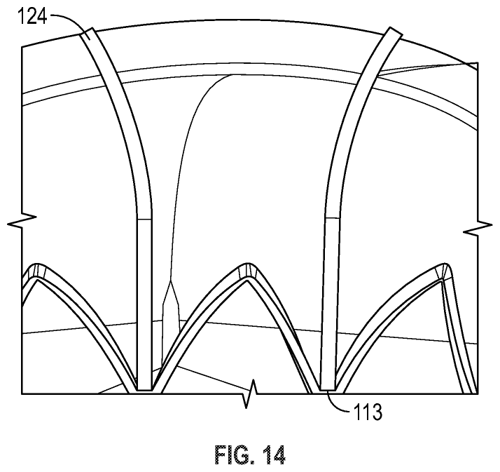

Various embodiments of minimal valve supports 124 are shown in FIGS. 12-13. For example, as shown in FIG. 12, the valve support 124 can extend from one or more longitudinal struts 113 and attach to the leaflets 16 at the nadir 96. As shown in FIG. 13, the minimal valve support 124 can be a hoop support that extends only along the inflow edge 95, but otherwise leaves the leaflet 14 unsupported within the inflow section. As shown in FIG. 14, the valve supports 124 can be wire forms that extend longitudinally from one or more longitudinal struts 113. The minimal valve supports 124 can advantageously help prevent partial prolapse of the leaflets 16 while still keeping the majority of the leaflets 16 in the inflow section unsupported.

In some embodiments, the minimal valve supports 124 (e.g., those shown in FIGS. 12-13) can be positioned between the leaflets 16 and a seal 177 (e.g., shown in FIG. 10A). Having the minimal valve supports 124 protected within the valve segment between the leaflets 16 and the seal 177 may advantageously making loading and/or releasing from the delivery system easier (e.g., by reducing friction and/or catching). Further, in some embodiments, the minimal valve supports 124 can be hinged at the connection to the frame 124 to assist in loading and/or releasing from the delivery system. In some embodiments, the minimal valve supports 124 that are positioned between the leaflets 16 and the seal 177 can be formed of a coil to help prevent kinking.

In some embodiments, the minimal valve supports 124 (e.g., those shown in FIGS. 12-13) can be at least partially laminated into a seal 177. In some embodiments, a seal support can be laminated into the seal 177 in addition to or in place of the minimal valve supports 124. The laminated seal 177 can, for example, include a polymeric material, such as a polyurethane.

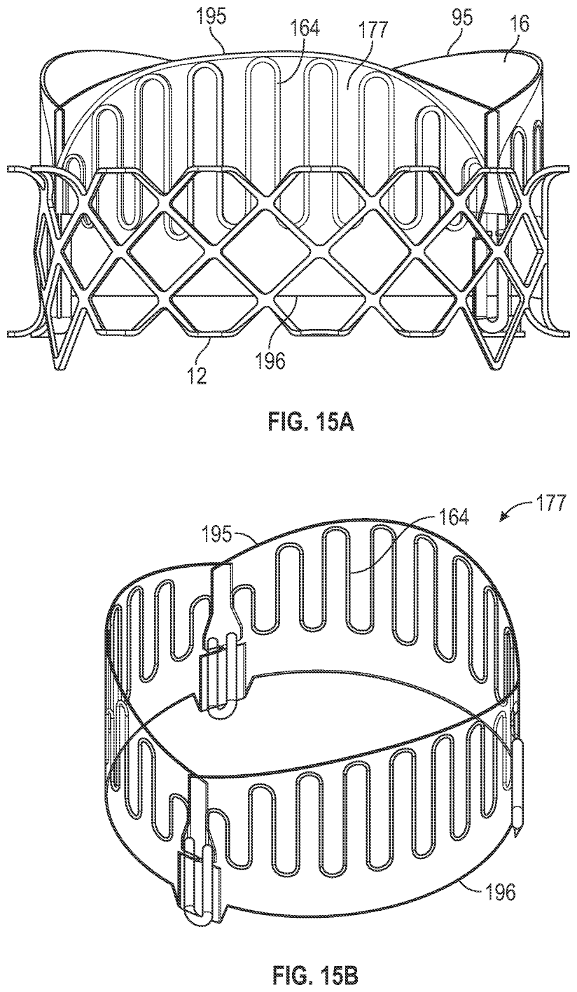

For example, FIGS. 15A-15B show a seal support 164 extending annularly within the seal 177. The seal 177 can have an inflow edge 195 that includes convex contours configured to match the inflow edge 95 of the leaflets 16 (e.g., three convex contours to match the three leaflets 16). Further, the seal support 164 can, for example, include an undulating or sinusoidal element or wireform, such as a nitinol wire, running through the seal 177. The undulating shape can advantageously enable the support 164 to be easily compressed during delivery of the valve prosthesis. In some embodiments, the shape of the seal support 164 can vary. For example, the undulating shape can include bulbous portions at the inflow or outflow ends of the undulating shape. As another example, the seal support 164 may include discrete axially-extending elements position within the seal 177.

As shown in FIGS. 15A-15B, the seal support 164 can extend all the way to or proximate to the inflow edge 195 of the seal 177. By having the seal support 164 extend close to the inflow edge 195, the seal support 164 can advantageously provide axially rigidity to the otherwise unsupported (or minimally supported) inflow edges 95 of the leaflets 16, thereby helping to prevent leaflet prolapse during valve opening. In some examples, the seal support 164 can extend closer to the inflow edge 195 of the seal 177 than the outflow edge 196 of the seal 177. Further, the seal support 164 can, in some embodiments, be entirely disconnected from the frame 12 of the valve prosthesis. In other embodiments the seal support 164 can be attached to the frame 12 (e.g., at the commissures).

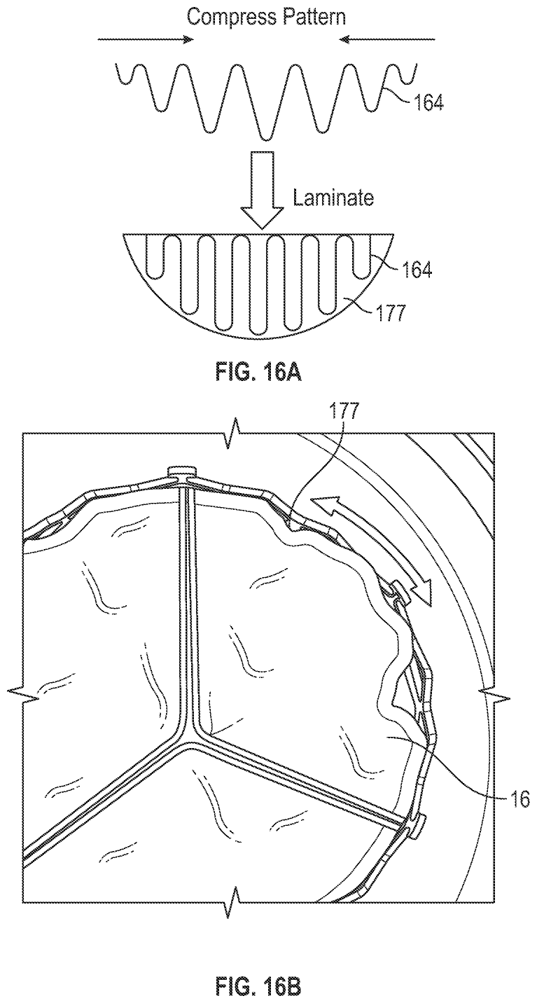

Referring to FIGS. 16A-16B, in some embodiments, the seal support 164 can be used to pretension the seal 177. That is, as shown in FIG. 16A, the undulating pattern of the seal support 164 can be compressed (shown by the arrows) and then laminated into the seal 177. Referring to FIG. 16B, as the seal support 164 expands, it will tension the seal 177 (as shown by the arrows). Tensioning the seal 177 with the seal support 164 can advantageously both reduce crumpling of the seal 177 and prevent leaflet prolapse during valve opening.

Referring to FIGS. 17A-17B, in some embodiments, a nadir support skirt 197 can extend between the inflow edge 195 of the seal 177 and the inflow portion 157 of the strut frame 12. That is, while the skirt 177 and frame 12 can remain spaced radially inwards from the inflow portions 157 at the inflow end 167 of the prosthesis, the nadir support skirt 197 can extend across the gap between the inflow edge 195 of the seal 177 and the inflow portion 157 of the strut frame 12. The nadir support skirt 197 can thus behave as a suspension between the leaflets 16 and the frame 12 at the inflow end 167 without requiring the leaflets 16 to conform to or be sewn directly to the frame 12. The nadir support skirt 197 can advantageously help prevent prolapse of the leaflets 16 during valve opening. Moreover, the nadir support skirt 197 can help prevent blood flow and/or coagulation in the gap between the leaflets 16 and the frame 12 at the inflow end 167 and/or can help prevent paravalvular leakage.

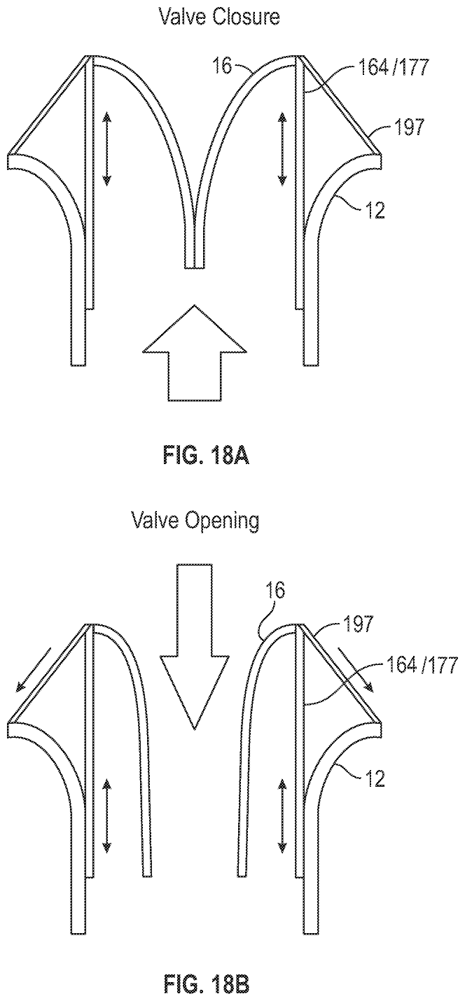

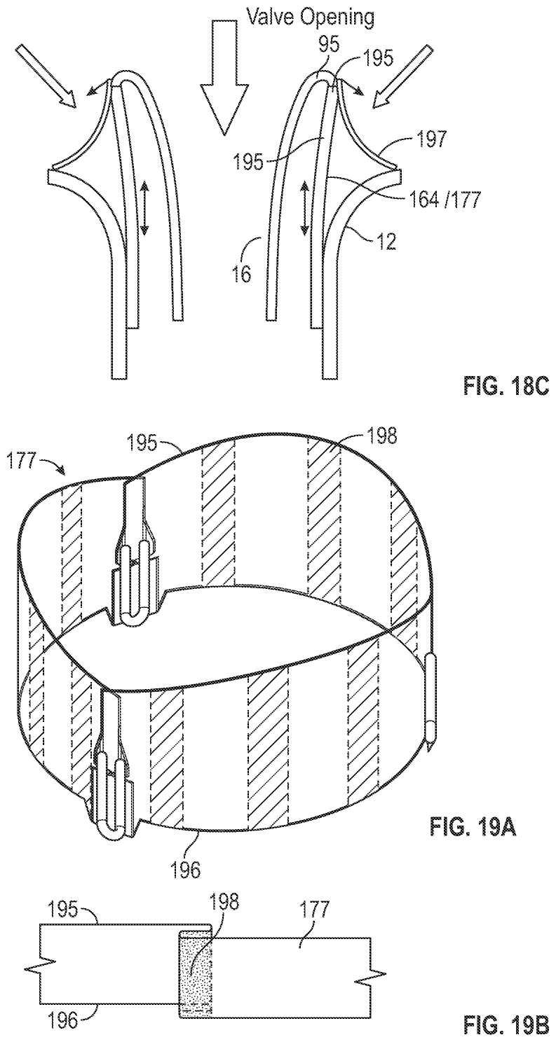

In some embodiments, referring to FIGS. 18A-18C, a valve prosthesis can include a combination of a seal support 164 and a nadir support skirt 197. During inflow (FIG. 18A), pressurization in the ventricle can ensure that the leaflets 16 remain closed and the seal 177 remains in tension. During outflow (FIG. 18B), the seal support 164 can ensures that the seal 177 remains in tension while the nadir support skirt 197 can pull radially outwards on the seal 177 and leaflets 16 to prevent prolapse. As shown in FIG. 18C, in some embodiments, the inflow edge 195 of the seal 177, and thus the inflow edge 95 of the leaflets 16, can tilt radially outwards during valve opening as a result of blood flow billowing the nadir support skirt 197 distally, thereby improving blood flow through the valve.

Referring to FIGS. 19A-19B, in some places, the seal 177 can include axial folds 198 or pleats that extend from the inflow edge 195 to the outflow edge 196. The folds 198 can be fixed in place, for example, with a polymer adhesive. The axial folds 198 can be positioned at various locations around the circumference of the seal 177. The axial folds 198 can act as a seal support to provide axial rigidity to the seal 177 while enabling easy collapse and sheathing for delivery.

In some embodiments, the inflow edge 95 of the leaflets can be entirely unsupported except at commissures of the leaflets 16. In some embodiments, the inflow edge of the leaflets 95 can be unsupported except at commissures of the leaflets 16 and the valve supports 124. In some embodiments, the axial folds 198, leaflet nadir support skirt 197, or seal support 164 can enhance the ability of the inflow edges 95 to remain unsupported by the frame 12 itself.

Referring to FIGS. 7A-7B, in some cases, the size of a valve prosthesis 10F (which can correspond to any of the valve prostheses 10 described herein), e.g., the magnitude of a frame height 137 of a valve prosthesis 10F in an expanded configuration) can be measured relative to one or more structures of the valve prosthesis 10F (e.g., a valve segment height of the valve prosthesis device in an expanded configuration, a leaflet height 174 when the device is expanded, and/or a diameter 139 of an expanded frame body) and/or relative to one or more biological structures (e.g., the mean diameter of a heart valve in which the device is deployed).

In some cases, the height 137 of a frame of the valve prosthesis 10F can be measured relative to the height 174 of a valve segment 14 of the valve prosthesis device 10F (e.g., valve segment height-to-frame height ratio, or VSTF ratio, e.g., a ratio of height 137 to height 174). In some cases, the height 174 of a valve segment 14 (or portion thereof, such as a valve leaflet) of an expanded valve prosthesis 10F is greater than the height of the frame of the valve prosthesis device (e.g., a VSTF ratio greater than 1).

A portion of frame structure 12, such as strut 113 and/or minimal valve support 124 (e.g., hoop structure) that can be used to provide frame structure 12 with compressive strength and/or resiliency can be made of a metal or a metal alloy. Representative examples of metals and metal alloys that can be used to form all or part of a portion of frame structure 12 include nickel-titanium alloys (NiTi), cobalt-chrome alloys, and stainless steel. A portion of a frame structure (e.g., strut 113 or minimal valve support 124) can be made of a material comprising one or more of the following metals: titanium, aluminum, cobalt, chrome, molybdenum, vanadium, zirconium, zinc, nickel, niobium, tantalum, magnesium, and iron. Specific titanium alloys that can be used include Ti-3Al-2.5V, Ti-5Al-2.5Fe, Ti-6-Al-4V, Ti-6Al-4V ELI, Ti-6Al-7Nb, Ti-15Mo, Ti-13Nb-13Zr, Ti-12Mo-6Zr-2Fe, Ti-45Nb, Ti-35Nb-7Zr-5Ta, and Ti-55.8Ni. A portion of a frame structure 12 can comprise a nickel-titanium alloy having equal or nearly equal amounts of nickel and titanium. For example, a nickel-titanium alloy can be 50 mol %, from 49.5 mol % to 50.5 mol %, from 49 mol % to 51 mol %, from 48.5 mol % to 51.5 mol %, from 48 mol % to 52 mol %, 47.5 mol % to 52.5 mol %, or from 47 mol % to 53 mol % nickel.

In some cases, a portion of valve prosthesis 10 can comprise a ceramic. For example one or more portions of frame structure 12 can comprise one or more of alumina, zirconia, quartz, pyrolytic carbon (e.g., pyrolytic carbon coated graphite), or a calcium phosphate such as hydroxyapatite.

A portion of valve prosthesis 10 can comprise a polymer (e.g., a sterilizable polymer and/or biocompatible polymer). In some cases, a polymer can comprise one or more of polyethylene (e.g., polyethylene terephthalate (PET), polytetrafluoroethylene (PTFE)), a fluoropolymer, silicone, polystyrene, nylon, polyurethane, thermoplastic polyurethane (TPU), polysiloxane, polylactide (PLA), polyglycolide (PGA), poly(lactide-co-glycolide) (PLGA), polycaprolactone (PCL) such as poly(ε-caprolactone), poly(methyl methacrylate), hyaluronan, polydioxanone, polyanhidride, or trimethylene carbonate. In some cases, a polymer of a valve prosthesis 10 or portion thereof can be a co-polymer (e.g., a block co-polymer). In some cases, a polymer can be cross-linked (e.g., using ultraviolet light) to increase strength and/or resiliency of a polymer.

Materials comprising valve prosthesis 10 or a portion thereof (e.g., frame structure 12, fabric covering 112, or strut 113) can be formed into solid structures or meshes. For example, fabric covering 112 can comprise one or more materials (e.g., polymers such as polyester or nylon) formed into a fabric or mesh.

In some cases, valve prosthesis 10 or a portion thereof (e.g., valve leaflet 16) can comprise a cell-based tissue. The use of a cell-based tissue as a material for valve prosthesis 10 or a portion thereof can offer various advantages, such as decreased thrombogenicity, improved integration of an implanted valve prosthesis 10 with surrounding native tissue, improved material properties of the device or portion thereof, and, in some cases, decreased immune response. For example, a valve prosthesis 10 (or portion thereof) comprising a cell-based tissue can exhibit mechanical properties closer to those of a healthy valve under static and/or dynamic mechanical loading. A cell-based tissue derived from a subject's own tissue (e.g., stem-cell derived tissues) or from an allogenic source comprising all or a portion of valve prosthesis 10 can decrease the likelihood of immunogenic response after implantation, in some cases. In some cases, one or more cells of a cell-based tissue useful in a valve prosthesis 10 can be autologous, allogeneic, or xenogeneic relative to a subject in which the prosthetic valve device is deployed. Representative examples of sources of one or more cells of a cell-based tissue useful in a valve prosthesis 10 are a human, a pig, or a cow. One or more distal (or ventricular) surfaces of leaflet 16 can be fabricated from, coated with, or treated with a biocompatible material.

As would be understood by a person of skill in the art, various embodiments of valve segments, valve anchors, and frame anchors, can offer advantages for the treatment or replacement of a native valve.

It should be understood that any feature described herein with respect to one embodiment can be substituted for or combined with any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for treating a diseased native valve in a patient, the device comprising:
a frame structure having an unexpanded configuration and an expanded configuration; and
a valve segment coupled to the frame structure, the valve segment comprising:
a plurality of leaflets, wherein an inflow edge of the plurality of leaflets is unsupported by the frame structure;
a seal attached to the inflow edge of the plurality of leaflets and positioned radially between the frame structure and the plurality of leaflets; and
a seal support attached to or within the seal, the seal support providing axial rigidity to the seal.

2. The device of claim 1, wherein the inflow edge of the plurality of leaflets is spaced radially inwards from an inflow edge of the frame structure when the frame structure is in the expanded configuration.

3. The device of claim 1, further comprising a nadir support skirt extending between the seal and an inflow edge of the frame structure.

4. The device of claim 1, wherein the inflow edge of the plurality of leaflets extends axially beyond an inflow edge of the frame structure such that the inflow edge of the plurality of leaflets extends further in an inflow direction than the inflow edge of the frame structure.

5. The device of claim 1, wherein the seal support is laminated within the seal.

6. The device of claim 1, wherein the seal comprises polyurethane.

7. The device of claim 1, wherein the seal support extends annularly within the seal.

8. The device of claim 1, wherein the seal support comprises an undulating wireform.

9. The device of claim 1, wherein the seal support extends proximate to the inflow edge of the valve segment.

10. The device of claim 1, wherein the seal support extends closer to an inflow edge of the seal than an outflow edge of the seal.

11. The device of claim 1, wherein the seal support is configured to pretension the seal.

12. The device of claim 1, wherein the seal support is disconnected from the frame structure.

13. The device of claim 1, wherein the seal support comprises a plurality of axial folds in the seal, each axial fold extending from an inflow edge of the seal to an outflow edge of the seal.

14. The device of claim 1, wherein the frame structure has a longitudinal length of less than 35 mm in the expanded configuration.

15. The device of claim 1, wherein the frame structure comprises a flared inflow section, a central annular section, and a flared outflow portion.

16. The device of claim 15, wherein the seal is attached to the central annular portion.

17. The device of claim 15, wherein the flared inflow section and flared outflow section are configured to engage an exterior anchor therebetween when the frame structure is in the expanded configuration.

18. The device of claim 1, wherein the leaflets of the plurality of leaflets are attached to the frame structure only at commissures of the leaflets.

19. The device of claim 1, wherein at least portion of the inflow edge of the plurality of leaflets extends axially beyond the frame structure while an entire outflow edge of the plurality of leaflets is positioned within the frame structure.

20-30. (canceled)
